(19) United States
(12) Patent Application Publication
(10) Pub. No.: US 2025/0241531 A1
(43) Pub. Date: Jul. 31, 2025

(54) PORTABLE WIDEFIELD FUNDUS CAMERA WITH HIGH DYNAMIC RANGE IMAGING CAPABILITY

(71) Applicant: THE BOARD OF TRUSTEES OF THE UNIVERSITY OF ILLINOIS, URBANA, IL (US)

(72) Inventors: Xincheng Yao, Chicago, IL (US); TaeYun Son, Chicago, IL (US); Alfa Rossi, Chicago, IL (US); Robison Chan, Chicago, IL (US)

(21) Appl. No.: 19/036,509

(22) Filed: Jan. 24, 2025

Related U.S. Application Data
(60) Provisional application No. 63/625,536, filed on Jan. 26, 2024.

(51) Int. Cl. A61B 3/12; A61B 3/00; A61B 3/14; G06T 5/50; G06V 10/60

(52) U.S. Cl. CPC A61B 3/12; A61B 3/0008; A61B 3/14; G06T 5/50; G06V 10/60; G06T 2207/20208; G06T 2207/20221

(57) ABSTRACT

Various examples are provided related to high dynamic range (HDR) imaging of a retina. In one example, a fundus camera includes an imaging sensor; an ophthalmic lens; and a source of input light. The source can illuminate the retina at sequentially increasing illumination power levels during a series of illumination periods within a pupillary reflex time of the eye. The imaging sensor can capture a corresponding low dynamic range (LDR) image of during each of the series of illumination periods, which can be combined to generate a HDR image of the retina. An external fixation target can be used to capture LDR images at different visual angles to generate HDR images, which can be combined to generate widefield HDR images.

### CROSS REFERENCE TO RELATED APPLICATIONS

This application claims priority to, and the benefit of, U.S. Provisional Application entitled “Portable Widefield Fundus Camera with High Dynamic Range Imaging Capability” having Ser. No. 63/625,536, filed Jan. 26, 2024, which is hereby incorporated by reference in its entirety.

### STATEMENT REGARDING FEDERALLY SPONSORED RESEARCH OR DEVELOPMENT

This invention was made with government support under awards P30 EY001792, RO1 EY023522, RO1 EY029673, RO1 EY030101, R01 EY030842, and R44 EY028786 from the National Eye Institute (NEI). The Government has certain rights in the invention.

### BACKGROUND

Vision-threatening eye diseases, such as diabetic retinopathy (DR), glaucoma, and age-related macular degeneration (AMD) affect approximately 400 million people worldwide, and the number of people with these conditions is projected to increase to 560 million by the year 2045. Early detection and prompt treatment assessment are important to prevent vision loss due to these diseases. Since eye conditions can affect both the peripheral and central retina, widefield fundus photography is important for the clinical management of eye diseases. Scanning laser ophthalmoscopy (SLO), such as Optomap (Optos, Marlborough, MA, USA) and Eidon (Icare USA Inc., Raleigh, NC, USA) systems can provide widefield and ultra-widefield fundus imaging by combing two or more laser wavelengths. However, sophisticated scanning and illumination devices needed for SLOs make them typically bulky and expensive. As almost 89% of visually impaired patients are from low- and middle-income countries (LMIC) with a lack of eye care providers to care for the population, a portable fundus camera to facilitate affordable telemedicine is preferable.

### SUMMARY

Aspects of the present disclosure are related to imaging systems comprising a fundus camera including high dynamic range (HDR) function. Fundus photography is indispensable for the clinical detection and management of eye diseases. Low image contrast and small field of view (FOV) are common limitations of conventional fundus photography, making it difficult to detect subtle abnormalities at the early stages of eye diseases. Further improvements in image contrast and FOV coverage are important for early disease detection and reliable treatment assessment. A portable, wide FOV fundus camera with high dynamic range (HDR) imaging capability is set forth. Miniaturized indirect ophthalmoscopy illumination can be employed to achieve the portable design for nonmydriatic, widefield fundus photography. Orthogonal polarization control can be used to eliminate illumination reflectance artifacts. With independent power controls, fundus images can be sequentially acquired and fused (using, e.g., an HDR imaging program) to achieve HDR function for local image contrast enhancement. A 101° eye-angle (67° visual-angle) snapshot FOV can be achieved for nonmydriatic fundus photography. The effective FOV can be expanded up to 190° eye-angle (134° visual-angle) with the aid of a fixation target without the need for pharmacologic pupillary dilation.

In one aspect, among others, a fundus camera comprises: a camera lens; an imaging sensor positioned on a first side of the camera lens; an ophthalmic lens positioned on a second side of the camera lens; and a source of input light, the source configured to direct the input light through the ophthalmic lens to illuminate a retina of an eye of a subject, the source configured to illuminate the retina at sequentially increasing illumination power levels during a series of illumination periods within a pupillary reflex time of the eye, the series of illumination periods comprising at least three illumination periods prior to the pupillary reflex time of the eye; where the imaging sensor is configured to capture a corresponding low dynamic range (LDR) image of output light reflected or backscattered by the retina during each of the series of illumination periods and received by the imaging sensor via the ophthalmic lens and camera lens. In various aspects, the sequentially increasing illumination power levels can be, such as, doubled in power at each subsequent illumination period in the series of illumination periods within the pupillary reflex time. The series of illumination periods can comprises at least two periods, such as, three periods, four periods, or more. The series of illumination periods can comprise three periods with the sequentially increasing illumination power levels at as, such as, 2 mW, 4 mW and 8 mW at the pupil plane for the subject. Each illumination period can be about 45 ms or less.

In one or more aspects, the source can be a source of linearly polarized input light with a polarization axis in a first direction, and the fundus camera can further comprise a quarter waveplate positioned on a side of the ophthalmic lens opposite the camera lens, the source configured to direct the linearly polarized input light to the quarter waveplate through the ophthalmic lens, the quarter waveplate converting the linearly polarized input light to circularly polarized input light that illuminates the retina at the increasing illumination levels during the series of illumination periods; and a linear polarizer disposed between the ophthalmic lens and the camera lens, the linear polarizer configured to receive depolarized output light reflected or backscattered by the retina and linearly polarized output light converted by the quarter waveplate from helically flipped output light reflected by the retina, and allow polarized output light with a polarization axis in a second direction that is aligned with the linear polarizer to reach the imaging sensor via the camera lens. The source of the linearly polarized input light can comprise a light source and a corresponding linear polarizer disposed between the light source and the ophthalmic lens, the light source and the camera lens positioned in a common plane with the light source offset from the camera lens by a buffer distance. The light source can be configured to provide near infrared (NIR) light and visible light. The visible light can be provided as the input light for illumination of the retina and the NIR light is provided for focus and guidance of the fundus camera.

In another aspect, a fundus camera system comprises the fundus camera and processing circuitry configured to generate a high dynamic range (HDR) image of the retina by combining the corresponding LDR images captured during the series of illumination periods prior to the pupillary reflex time of the eye. In various aspects, intensity values of pixels of the corresponding LDR images can be combined based at least in part upon a weighting function associated with each pixel. The processing circuitry can be integrated in the fundus camera or remotely located from the fundus camera. In one or more aspects, the fundus camera system can further comprise an external fixation target visible to another eye of the subject. The external fixation target is repositionable with respect to the fundus camera.

In another aspect, a method for high dynamic range (HDR) imaging of a retina comprises directing input light from a source positioned on a first side of an ophthalmic lens to illuminate a retina of an eye on a second side of the ophthalmic lens, the retina illuminated at sequentially increasing illumination power levels during a series of illumination periods within a pupillary reflex time of the eye, the series of illumination periods comprising at least three illumination periods prior to the pupillary reflex time of the eye of a subject; capturing, by an imaging sensor, a corresponding low dynamic range (LDR) image of output light reflected or backscattered by the retina during each of the series of illumination periods and received by the imaging sensor via the ophthalmic lens and a camera lens positioned on the first side of an ophthalmic lens; and generating, by processing circuitry, a high dynamic range (HDR) image of the retina by combining the corresponding LDR images captured during the series of illumination periods prior to the pupillary reflex time of the eye. In one or more aspects, the sequentially increasing illumination power level can be, such as, doubled in power at each subsequent illumination period in the series of illumination periods within the pupillary reflex time. The retina can be illuminated for a fixed time period during each illumination period. Intensity values of pixels of the corresponding LDR images can be combined based at least in part upon a weighting function associated with each pixel

In various aspects, the method can comprise directing near infrared (NIR) light from the source to illuminate the retina; and focusing and aligning the imaging sensor for capture of the corresponding LDR images prior to directing the input light from the source. The method can further comprise positioning an external fixation target at a location visible to another eye of the subject, the location at a first visual angle away from a macula centered image; with the other eye focused on the fixation target, directing input light from the source to illuminate the retina at the sequentially increasing illumination power levels during a second series of illumination periods within the pupillary reflex time of the eye, the second series of illumination periods comprising at least three illumination periods prior to the pupillary reflex time of the eye; capturing, by the imaging sensor, a corresponding LDR image of output light reflected or backscattered by the retina during each of the second series of illumination periods; generating, by the processing circuitry, a second HDR image of the retina by combining the corresponding LDR images captured during the second series of illumination periods prior to the pupillary reflex time of the eye; and generating a widefield HDR image by combining the HDR image of the retina with the second HDR image. The method can further comprise repositioning the external fixation target at another location visible to the other eye of the subject, the other location at a second visual angle away from the macula centered image opposite the first visual angle; with the other eye focused on the fixation target, directing input light from the source to illuminate the retina at the sequentially increasing illumination power levels during a third series of illumination periods within the pupillary reflex time of the eye, the third series of illumination periods comprising at least three illumination periods prior to the pupillary reflex time of the eye; capturing, by the imaging sensor, a corresponding LDR image of output light reflected or backscattered by the retina during each of the third series of illumination periods; generating, by the processing circuitry, a third HDR image of the retina by combining the corresponding LDR images captured during the third series of illumination periods prior to the pupillary reflex time of the eye; and generating an ultra-widefield HDR image by combining the HDR image of the retina with the second and third HDR images.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A and 1B schematically illustrate examples of conventional transpupillary illumination with an annular pattern in a traditional fundus camera and miniaturized indirect illumination with a simplified single-point pattern, in accordance with various embodiments of the present disclosure.

FIGS. 2A and 2B schematically illustrate an example of an optical imaging system for imaging of a retina, in accordance with various embodiments of the present disclosure.

FIG. 2C is an image showing an example of a prototype fundus camera used during an imaging operation, in accordance with various embodiments of the present disclosure.

FIGS. 2D and 2E schematically illustrate examples of a back reflection pattern at the camera sensor and a fundus image with back reflection artifact (marked with white arrow), in accordance with various embodiments of the present disclosure.

FIG. 3 schematically illustrates an example of a high dynamic range (HDR) imaging principle, in accordance with various embodiments of the present disclosure.

FIG. 4A is an example of a near infrared (NIR) guidance image, in accordance with various embodiments of the present disclosure.

FIG. 4B-4D are examples of low dynamic range (LDR) images using low power, medium power, and high power flash, in accordance with various embodiments of the present disclosure.

FIG. 4E is an HDR image composed of the LDR images of FIGS. 4B-4D, in accordance with various embodiments of the present disclosure.

FIG. 5A includes LDR images of an eye diagnosed with DR with different illumination power levels, in accordance with various embodiments of the present disclosure.

FIG. 5B is an HDR image of the eye from the LDR images of FIG. 5A, in accordance with various embodiments of the present disclosure.

FIGS. 5C and 5D are cropped portions marked with squares in the LDR and HDR images of FIGS. 5A and 5B, in accordance with various embodiments of the present disclosure.

FIGS. 6A and 6B are an HDR image of a patient diagnosed with diabetic retinopathy and an image from Volk Pictor Plus from the same subject, in accordance with various embodiments of the present disclosure.

FIG. 6C are hot colormaps of the cropped regions marked with squares in FIGS. 6A and 6B, in accordance with various embodiments of the present disclosure.

FIGS. 7A and 7B are an HDR image of a patient diagnosed with age-related macular degeneration and an image from Volk Pictor Plus from the same subject, in accordance with various embodiments of the present disclosure.

FIG. 7C are hot colormaps of the cropped regions marked with squares in FIGS. 7A and 7B, in accordance with various embodiments of the present disclosure.

FIG. 8A is an example of an ultra-widefield HDR image produced by merging seven HDR images together, in accordance with various embodiments of the present disclosure.

FIG. 8B is an example of an image taken with Optomap, in accordance with various embodiments of the present disclosure.

FIG. 9 is a schematic diagram illustrating an example of processing or computing circuitry for implementing HDR imaging, in accordance with various embodiments of the present disclosure.

### DETAILED DESCRIPTION

Disclosed herein are various examples related to imaging systems comprising a fundus camera including high dynamic range (HDR) function. In some embodiments, the imaging system can comprise a portable widefield fundus camera with nonmydriatic HDR. The system can comprise miniaturized indirect ophthalmoscopy illumination to enable the portable design for widefield fundus photography. Near infrared (NIR) light can be used for image focusing and guidance. Systems herein can comprise pulsed visible light illumination with flexible power control components wherein rapid, nonmydriatic imaging is detectable within a time window before visible light illumination caused pupillary response. Other embodiments are contemplated as well. Reference will now be made in detail to the description of the embodiments as illustrated in the drawings, wherein like reference numbers indicate like parts throughout the several views.

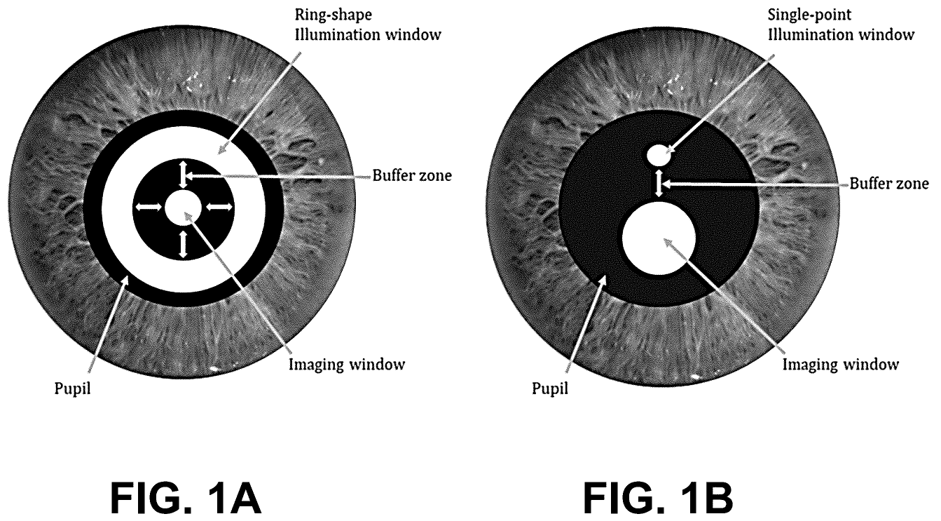

Currently available commercial portable fundus cameras have a limited field of view (FOV) and image contrast, which are the common limitations of annular trans-pupillary illumination. The FOV of conventional fundus cameras is typically around 45°-67.5° eye-angle (30°-45° visual angle). In conventional fundus cameras, the illumination light is delivered through the annular shaped peripherical region of the available pupil, and the imaging light from the back of the eye is collected through the center of the pupil. FIG. 1A illustrates an example of conventional transpupillary illumination with an annular pattern in a traditional fundus camera. At the pupil plane, a proper buffer zone between the illumination and imaging windows is required to prevent reflection artifacts from the cornea and crystalline lens. At the retina plane, the region for imaging must be fully covered by the illumination light delivered through the annular-shaped (or ring-shaped) window. Therefore, the regions used for light illumination and imaging observation should be carefully balanced. This tradeoff limits the FOV, thus pharmacologic pupillary dilation, which is stressful for both patient and operator, is often needed to expand the FOV for comprehensive eye examination.

Another common challenge of fundus photography is that the luminescence range needed to capture sufficient details in the human fundus may exceed the dynamic range of the camera sensors. For example, the light reflected from the optic nerve head is multiple orders greater than that of the macular region due to the different density of the pigmented cells and nerve fibers. Therefore, when the illumination power is adjusted to image the peripapillary region, the macular region falls below the noise floor of the camera sensor. Adjusting the power level for better macula imaging often leads to saturation at the peripapillary region. The structural details residing below the noise floor or in the saturated region cannot be recovered. As the compromised image contrast and inhomogeneity can hinder grading of retinal disease severely, a more holistic image is preferable. A plethora of research has been performed to study fundus image quality improvement by correcting scene inhomogeneity or enhancing the contrast between retinal segments. These algorithms perform poorly when preserving details in saturated regions and can often create false colors that cause misdiagnosis.

FIG. 1B illustrates an example of miniaturized indirect illumination with a simplified single-point pattern. Miniaturized indirect illumination provides a simple workaround for the limitation of the available pupil, making it an alternative solution for developing widefield portable fundus cameras. In accordance with the principles herein, a miniaturized indirect illumination-based fundus camera is set forth to achieve portable, nonmydriatic, widefield fundus imaging. Orthogonal polarization control is used to eliminate the back reflected light artifacts encountered in miniaturized indirect illumination-based fundus systems. High dynamic range (HDR) imaging capability is integrated to increase the dynamic range and enhance the contrast of fundus image for better visualization of the retina and retinal disease biomarkers.

In fundus imaging, the conventional unit for FOV evaluation is the visual-angle degree. However, recently emerging widefield fundus imagers such as Optomap (Optos Inc., Marlborough, MA, USA), ICON (Neolight, Pleasanton, CA, USA) and Retcam (Natus Medical Systems, Pleasanton, CA, USA) are using eye-angle degree as the unit of measurement. For easy comparison, both visual-angle and eye-angle values are provided herein.

### Materials and Methods

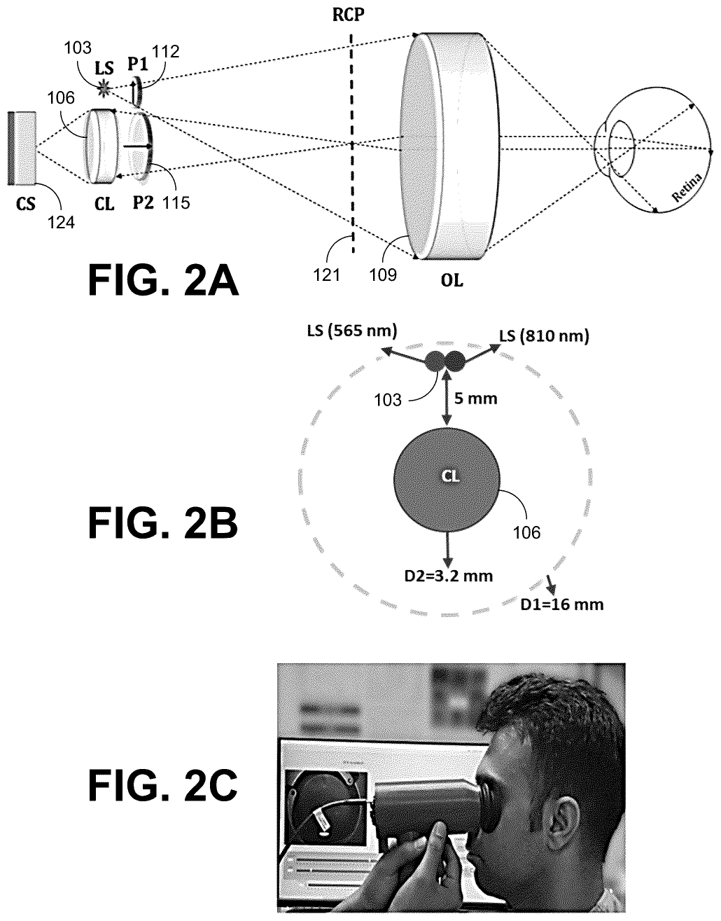

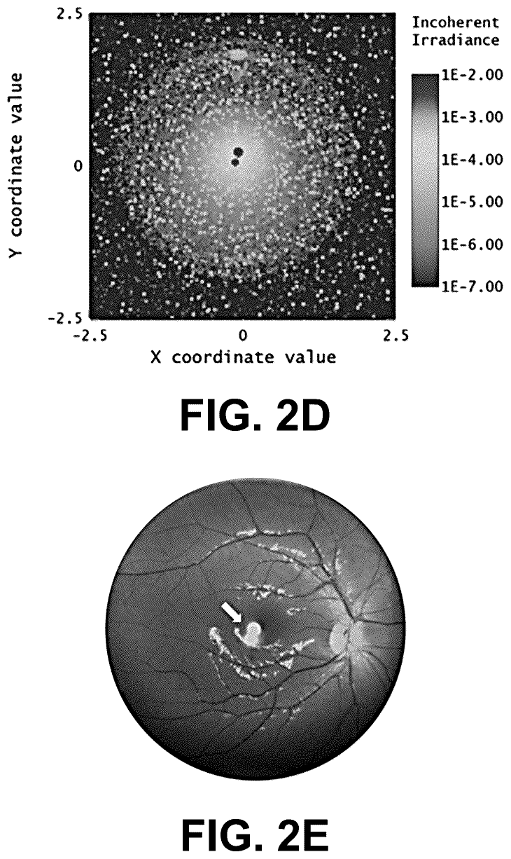

Experimental setup. FIGS. 2A and 2B illustrate the optical layout of the imaging system and FIG. 2C is a representative photograph of the system during imaging. The camera lens (CL) 106 (8 mm f/2.5 micro video lens, 58-203, Edmund Optics Inc., Barrington, NJ) and light source (LS) 103 were situated on the same plane which was conjugated to the pupil of the eye. The ophthalmic lens (OL) 109 (25 mm focal length) creates an image of the retina at the retina conjugate plane (RCP) 121 which is relayed to the camera sensor (CS) 124 (FL3-U3-120S3C-C, FLIR Integrated Imaging Solutions Inc., Richmond, Canada). The magnification from the eye pupil to the CL-LS plane is set to be 4× and the retina to the sensor is 0.22×. Assuming the pupil diameter is 4 mm at room light condition without pupillary dilation, this corresponds to a 16 mm diameter region available in the CL-LS plane for the camera lens 106 and light source 103 to be located in (layout separately shown in FIG. 2B). The light source 103 contains an 810 nm LED (M850LP1, Thorlabs Inc., Newton, NJ, USA) for near infrared (NIR) imaging guidance and a broadband (FWHM 104 nm) LED with center wavelength at 565 nm (M565D2, Thorlabs Inc., Newton, NJ, USA) for color fundus imaging. The 565 nm illumination is optimal for visualizing retinal vasculature, differentiating the arterioles and the venules. Additional details regarding the imaging system can be found in U.S. patent application Ser. No. 18/800,588, entitled “Preserving Polarization Maintaining Photons for Enhanced Contrast Imaging of the Retina,” filed Aug. 12, 2024, which is hereby incorporated by reference in its entirety.

For understanding the back-reflected light distribution without polarization control, non-sequential ray tracing was implemented with Zemax OpticStudio 18.7 (ZEMAX LLC., Kirkland, WA, USA). FIG. 2D shows the reflectance artifact pattern at the camera sensor 124. There are two bright spots at the center with maximum intensity, as well as reflected light rays distributed throughout the whole sensor plane. Hence, apart from having two saturated spots at the center of the image, there would be an overall haze in the fundus image if the back-reflected light is not rejected. FIG. 2E shows a fundus image taken without any polarization control where the reflectance artifacts and overall haze could be observed. Hence, orthogonal polarizers P1 112 and P2 115 (LPVISE050-A, Thorlabs Inc., Newton, NJ, USA) are set in front of the light source 103 and camera lens 106 in FIG. 2A to achieve cross polarized light illumination and detection to remove back reflected light from the ophthalmic lens 109 surface facing the camera lens 106.

High dynamic range imaging. In signal theory, the dynamic range is expressed as the ratio of the largest measurable signal to the smallest measurable signal. In the case of imaging, the signal is the light reflected from any surface and falling onto the sensor 124. The sensor 124 is composed of pixels, and each pixel generates an electronic signal proportional to the number of photons falling into it. If the amount of light on a pixel is equal to its full well capacity (Qₘₐₓ), the pixel is saturated, and a further increase in light intensity will not change the output value. Similarly, there is a minimum amount of energy (Q*ₘᵢₙ) that must fall onto a pixel to generate any signal. Moreover, due to sensor's inherent noise, the actual minimum energy that causes perceptible signal output is higher, referred to herein as Qₘᵢₙ. Therefore, the dynamic range of the sensor is:

$$D = \frac{{Q}_{max}}{{Q}_{min}} . \qquad ( 1 )$$

It is evident that, if part of a scene reflects less energy than Qₘᵢₙ or more energy than Qₘₐₓ, the sensor will not be able to capture those details. The actual dynamic range is not dependent on the number of bits in the image created by the sensor but determined by the pixel size and the material property of the sensor. Since a tradeoff is inevitable among the sensor resolution, pixel size, and imaging speed, the dynamic range of a digital camera is limited. HDR imaging is a technique to extend the dynamic range of an imaging system beyond the dynamic range of the digital camera sensor.

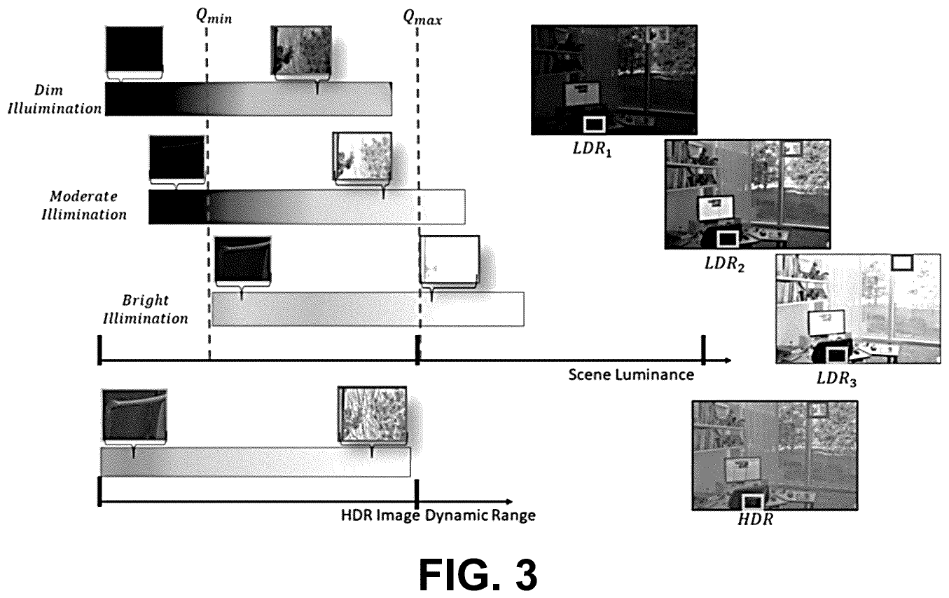

FIG. 3 illustrates the basic principle of the HDR imaging. The horizontal bars show the luminescence range of the scene in dim, moderate and bright illumination conditions. LDR₁, LDR₂ and LDR₃ are the corresponding images of the scene. In dim illumination condition, the region marked by the square in LDR₁ is below the noise floor, thus the details are imperceptible. By increasing the scene illumination, the brightness of this region could be lifted above the noise floor, shown in LDR₃. However, as the scene illumination is increased, regions which were bright in LDR₁, could go above the saturation, depicted by the region marked with the square in LDR₃. It is evident that, combined LDR₁, LDR₂ and LDR₃ improved the visualization of details present in the scene, although individually they show compromised visualization due to light saturation or background noise. The HDR image combines the information preserved by the image-set and produces a composite image containing crucial details in both highlights and shadows. An HDR imaging program can be used to combine or fuse the LDR images.

If N images of a scene are taken with N different illumination conditions, then the intensity value for each pixel of HDR image Lᵢⱼ can be estimated using this formula:

$${L}_{ij} = {\sum }_{k = 1}^{N} \frac{{f}^{- 1} ( {z}_{ij} ) w ( {z}_{ij} )}{Δ x} / {\sum }_{k = 1}^{N} w ( {z}_{ij} ) , \qquad ( 2 )$$

where zᵢⱼ is the pixel value of the LDR images and w(zᵢⱼ) is the weighting function associated with that pixel. These weights determine how much each LDR pixel would contribute to the corresponding HDR pixel. The camera response function is denoted as f. LDR images could be taken by varying the illumination parameters, such as exposure time, flash power, etc. Ax denotes the parameter that is varied, and each pixel of an LDR image should be divided by the respective parameter value (exposure time or the power used to take that image) for normalization. The weighting function can be calculated from the camera response function (see, e.g., “On being ‘undigital’ with digital cameras: extending dynamic range by combining differently exposed pictures” by S. Mann and R. Picard (Proc. IS & T Annual Meeting, vol. 48, March 1996), “Radiometric self calibration” by T. Mitsunaga and S. K. Nayar (in Editor (Ed.)\hat{ }(Eds.): ‘Book Radiometric self calibration’ (1999, edn.), pp. 374-380, Vol. 371), and/or “Recovering high dynamic range radiance maps from photographs” by P. E. Debevec and J. Malik (ACM SIGGRAPH 2008 classes' (2008), pp. 1-10)).

Theoretically, increasing the number of LDR images would reduce the effect of sensor noise and thus increase the details preserved. But for nonmydriatic fundus imaging, the total acquisition time should be below the pupillary reflex time, which is around 150 ms. Therefore, three images of the eye were taken, each with 35 ms exposure time, and varied the illuminating power. Other exposure times can be used, e.g., 45 ms or less, 40 ms or less, 35 ms or less, 30 ms or less, 25 ms or less, or intermediate exposure times as can be understood by one of skill in the art. The rationale of changing the illumination power instead of exposure time is as follows. First, there is a hardware overhead delay when the exposure setting is changed between two acquisitions. Second, the long exposure time needed to get the high intensity LDR image causes motion blur in some cases. The power levels for three images were experimentally set after calibrating with subjects from different eye pigmentation levels. Increasing the illumination power by a factor of two with each acquisition worked the best for this study. With this configuration, all the shadow, midtone and highlight information were reasonably preserved in the HDR images.

Human subjects and imaging protocol. This study was approved by the Institutional Review Board of the University of Illinois at Chicago and followed the ethical standards stated in the Declaration of Helsinki. Informed consent was obtained from each subject before the experiment. It was confirmed that none of the subjects had any history of seizure since the experiment involved bright flashes of light.

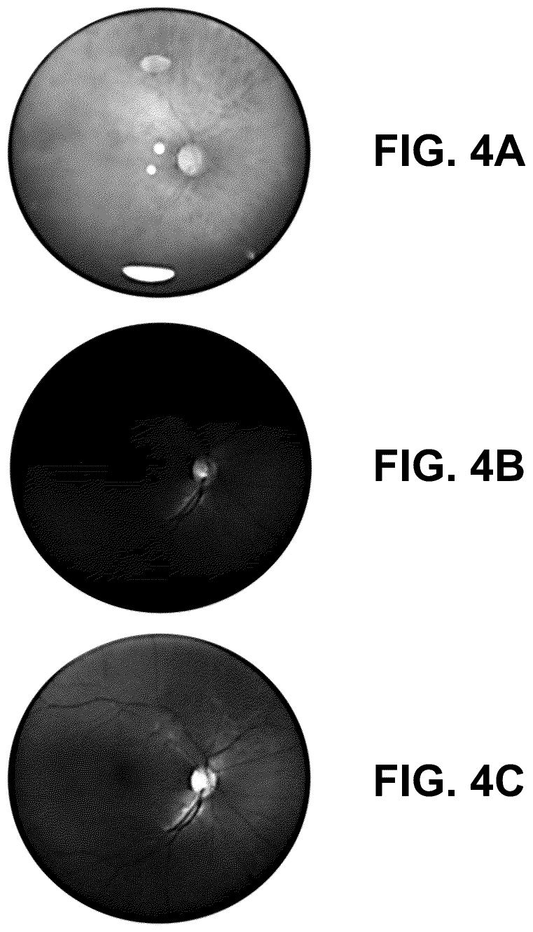

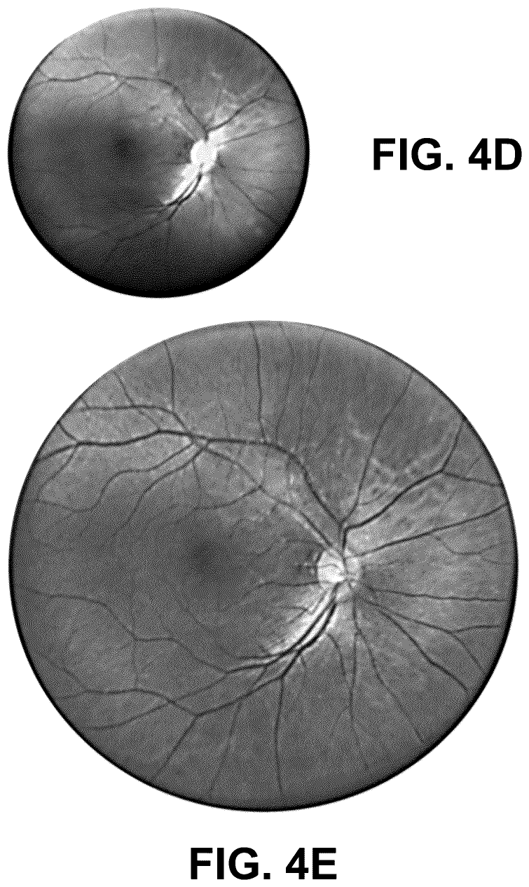

The minimum required pupil size was 4 mm for the system, which is readily available in dimly lit room conditions. The alignment of the system and focusing was done using NIR light as shown in FIG. 4A, so it did not stimulate the pupillary reflex. After focusing, three sequential visible light images were taken with the illumination power being doubled with each acquisition (e.g., 2 mW, 4 mW and 8 mW at the pupil plane for the subject as shown in FIGS. 4B-4D) and with an exposure time of 35 ms for each image. A LabVIEW interface was created to stream the live view of the alignment procedure and to take sequential images.

After capturing the image sequence, HDR images were created by a custom-built MATLAB program. FIG. 4E shows an example of a composed HDR image. In order to compare the quality of the images taken by the device, fundus images were taken afterwards with a commercially available portable fundus imaging device Pictor Plus (Volk, Mentor, OH, USA). For FOV extension, a dimly lit external LED fixation target was placed in front of the subjects, and they were instructed to follow the target with the eye that was not being imaged. After taking a fundus image with the macula at the center, two images were captured by changing the image location approximately 30°-33° visual-angle (45°-50° eye-angle) away from the macula centered image in both left and right directions. Afterwards, four images were taken by changing the macula centered image location approximately 10°-15° visual-angle (15°-22.5° eye-angle) diagonally.

Light safety. ISO standard “Ophthalmic Instruments-Fundus Cameras” (10940:2009) was used to quantitatively calculate the ocular safety of the retina against photochemical hazards. The ISO standard allowed 10 J/cm² radiant exposure on the retina which is 10 times lower than the retinal photochemical damage threshold. It also provides photochemical hazard weighting function to calculate the weighted irradiance. Any light scattered by the cornea or the crystalline lens were neglected and assume all the lights are falling on the cornea and reach the retina. For visible light, the weighted irradiance of each flash was calculated by considering the spectrum of the LED and the photochemical hazard function. Afterwards, the products of weighted irradiance and exposure time of all three flashes were added to get the radiant exposure on the retina for each acquisition. The radiant exposure of each acquisition was calculated to be 4.71×10⁻⁶ J/cm², which is well below the safety limit.

For the NIR guidance illumination, weighted irradiance was calculated to be 0.06 mW/cm². The permitted time for continuous guidance using NIR wavelength was calculated with this formula:

$${T}_{max} = \frac{Maximum allowed weighted irradiance}{Calculated weighted irradiance} . \qquad ( 3 )$$

The maximum exposure time for continuous illumination with NIR light is 46.3 hours.

### Results

FIGS. 4A-4D illustrate the imaging procedure done on a healthy subject. The NIR guidance is illustrated in FIG. 4A. As the design wavelength of the polarizer is in the visible light region, the NIR image had back-reflection artifacts. Since NIR wavelength was exclusively used for guidance, these reflection artifacts were not an issue for this step. Three LDR images taken with three illumination power levels are shown in FIGS. 4B-4D. The visible light images are free of any reflection artifacts.

Details in different fundus regions are preserved in different images of the image set. For example, the low power image shown in FIG. 4B preserves the information at the optic disc, but the other regions are barely recognizable. The LDR image taken with medium power level in FIG. 4C preserves the structural details of the nerve fibers around the optic disc. And finally, the high power LDR image in FIG. 4D preserves the details near the macula and the periphery, but the optic disc is saturated. Notably, none of the LDR images are holistic and each excludes crucial details of the retina which are irrecoverable since they are either near the noise floor or saturation. FIG. 4E is the HDR image created from the LDR images. All of the above-mentioned information contained in the set of LDR images is preserved in the single HDR image.

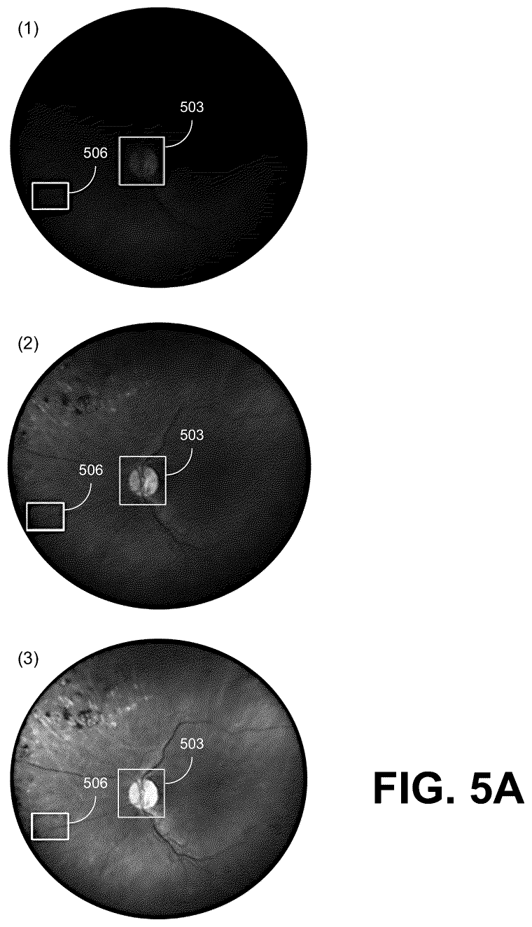

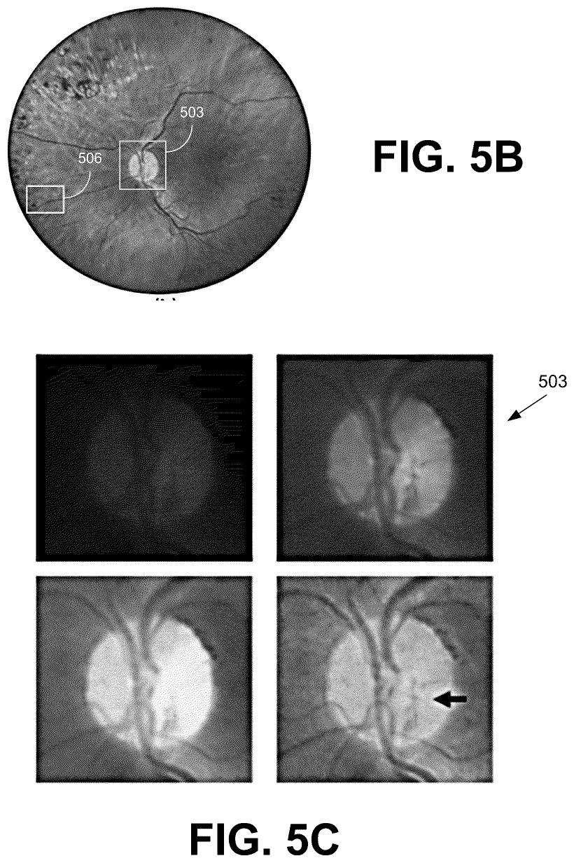

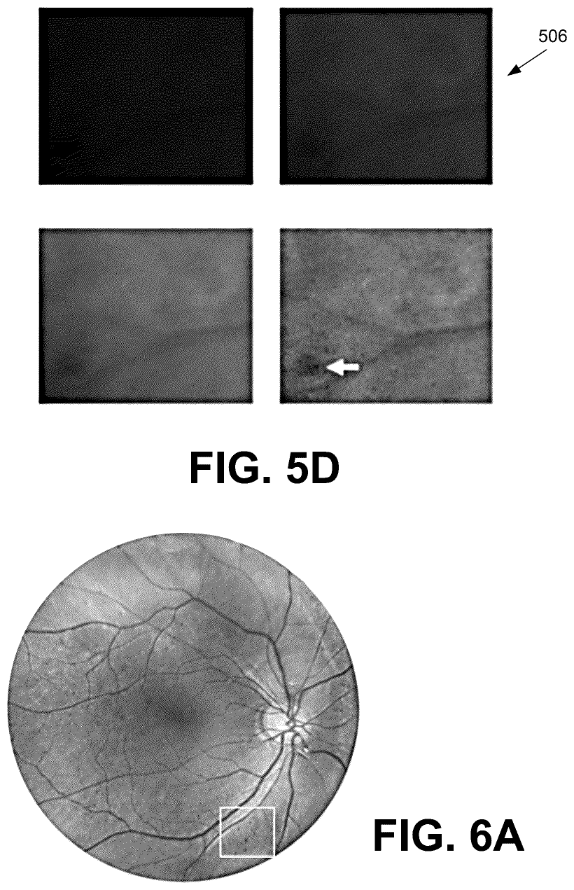

FIGS. 5A and 5B show the LDR images and the HDR image from a patient diagnosed with DR. A small section around the optic disc (marked with square 503) was cropped from the LDR and HDR images, shown in FIG. 5C. Similarly, another more peripheral (marked with square 506) region was also selected, shown in FIG. 5D. It is evident from FIGS. 5C and 5D that the HDR image preserves the information of small vessel growth (neovascularization, marked with a black arrowhead) overlying the optic disc, as well as microaneurysms (marked with a white arrowhead) present in the periphery, giving the clinicians a comprehensive understanding of the stage of DR in this patient. None of the LDR images preserved both of this information at the same time.

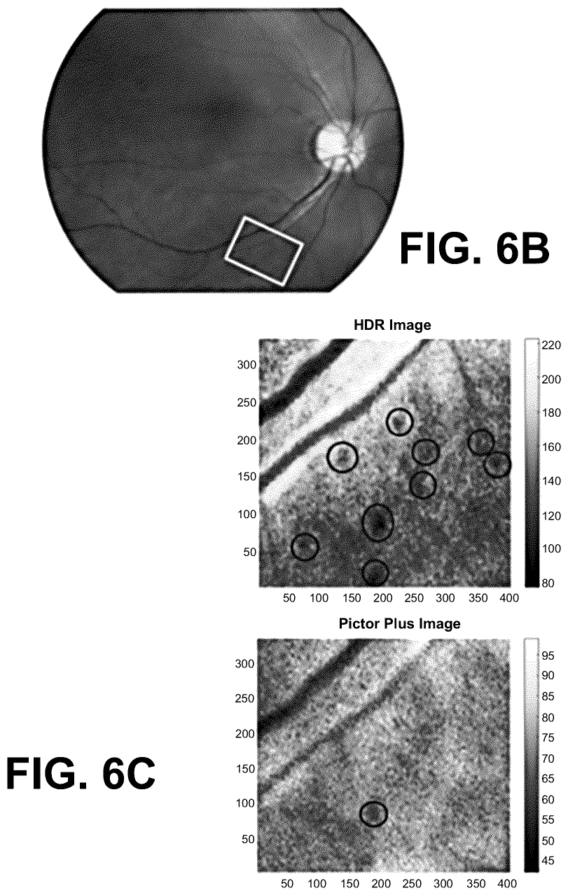

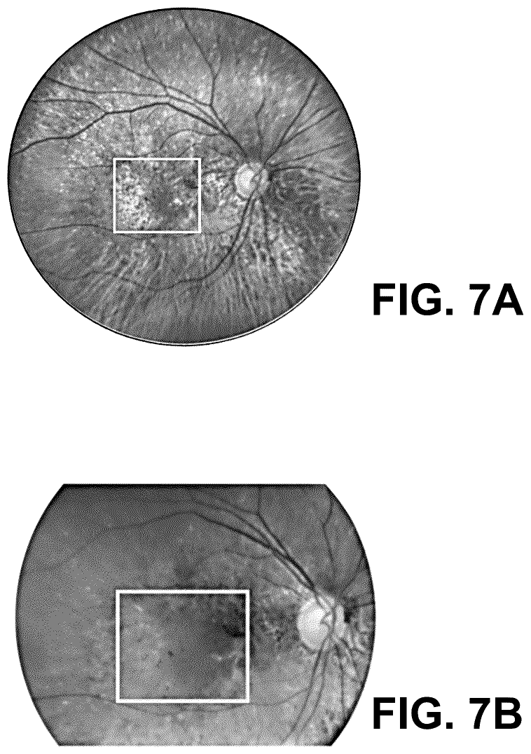

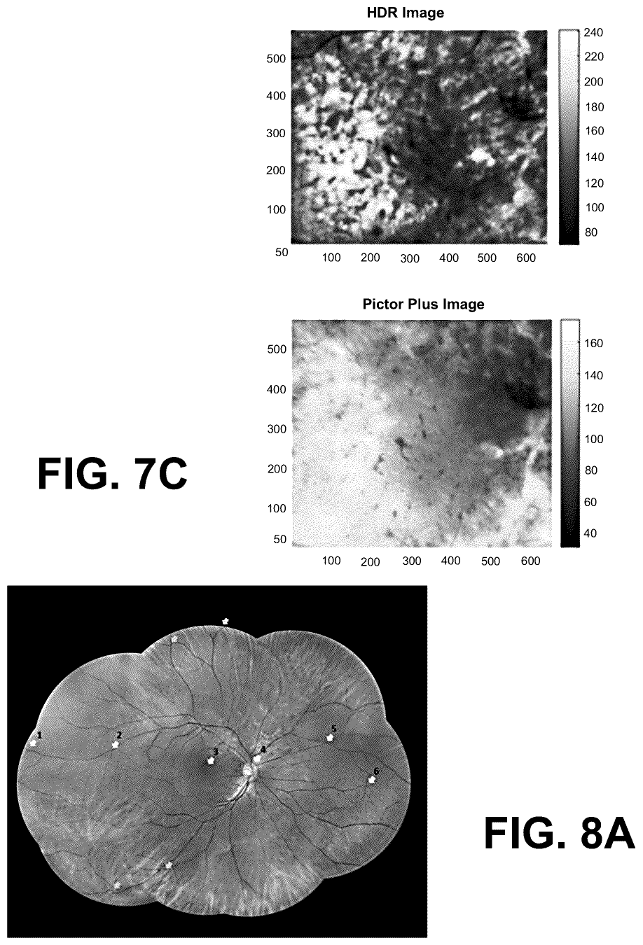

For comparative evaluation, FIGS. 6A-6C and FIGS. 7A-7C show HDR images captured with the prototype system (FIGS. 2A-2C) and a commercial portable fundus camera Volk Pictor Plus (Volk, Mentor, OH) from patients diagnosed with DR and AMD, respectively. A small portion of the fundus showing microaneurysms were cropped from FIGS. 6A and 6B and the corresponding hot colormaps are presented in FIG. 6C. It is evident from the hot colormaps that the contrast of the microaneurysms (marked with black circles) is much better in the image taken with the prototype system. In a similar manner, a section around the macula of a patient diagnosed with AMD was cropped (FIGS. 7A-7C). The pigment clumping (black spots) and RPE atrophy (white granular structures) are clearly more recognizable in the HDR image, evident from the respective hot colormaps.

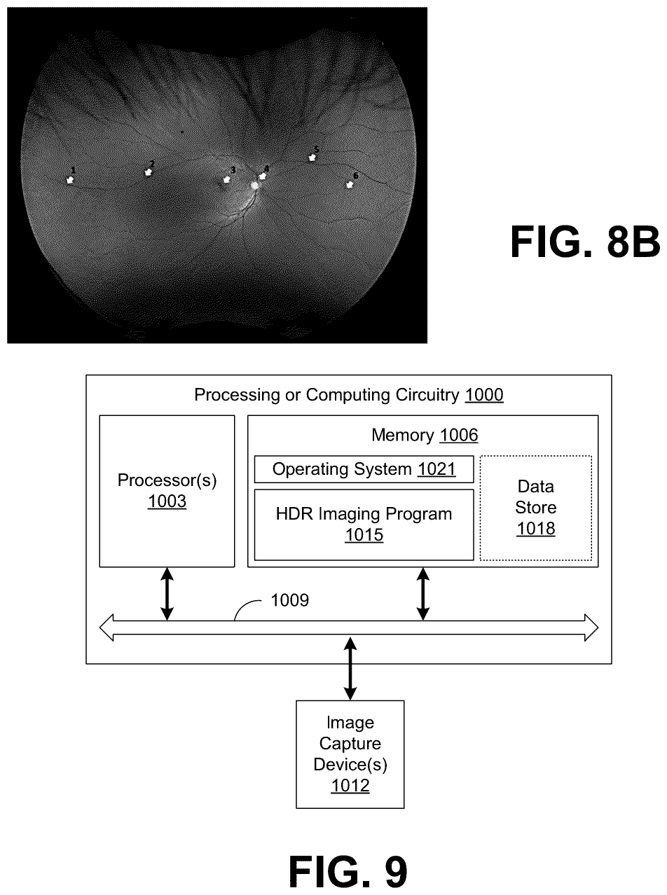

To demonstrate the capability of imaging the peripheral retina, seven images were taken with the aid of an external fixation target and merged together (see FIG. 8A) using, e.g., an HDR imaging program. A comparative image taken with ultra-widefield SLO Optomap is shown in FIG. 8B. The FOV of the image in FIG. 8A is estimated to be 190° eye-angle (134° visual-angle) horizontally and 146° eye-angle (100° visual-angle) vertically. From FIGS. 8A and 8B, it is evident that all major features present in the horizontal direction (marked with the white arrows 1-6 in both images) of the image taken with the Optomap SLO are also preserved in the image taken with the portable HDR fundus camera. In the vertical direction, the presence of eyelashes can block the laser scanning light of the Optomap. Without the scanning requirement, the snapshot HDR fundus camera is not affected by the eyelashes, and thus can image the peripherical structures (see upper arrows in FIG. 8A) beyond the accessible region of the Optomap (see FIG. 8B). The subjects of FIGS. 4A-4E and FIGS. 8A-8B are Asian, and the rest of the images are from subjects of Hispanic racial background.

With reference to FIG. 9, shown is a schematic block diagram illustrating an example of processing or computing circuitry 1000. In some embodiments, among others, the processing or computing circuitry 1000 may include a processing or computing device such as, e.g., a smartphone, tablet, computer, etc. As illustrated in FIG. 9, the processing or computing circuitry 1000 can include, for example, a processor 1003 and a memory 1006, which can be coupled to a local interface 1009 comprising, for example, a data bus with an accompanying address/control bus or other bus structure as can be appreciated. To this end, the processing or computing circuitry 1000 may comprise, for example, at least one server computer or like device, which can be utilized in a cloud-based environment. In some embodiments, the processing or computing circuitry 1000 can include one or more network interfaces that may comprise, for example, a wireless transmitter, a wireless transceiver, and a wireless receiver. The network interface can communicate to a remote computing device using, e.g., a Bluetooth protocol or other wireless protocol.

In some embodiments, the processing or computing circuitry 1000 can include one or more network/communication interfaces. The network/communication interfaces may comprise, for example, a wireless transmitter, a wireless transceiver, and/or a wireless receiver. As discussed above, the network interface can communicate to a remote computing device using a Bluetooth, WiFi, or other appropriate wireless protocol. As one skilled in the art can appreciate, other wireless protocols may be used in the various embodiments of the present disclosure. In addition, the processing or computing circuitry 1000 can be in communication with one or more image capture device(s) 1012 such as, e.g., an optical imaging system and/or fundus camera of FIGS. 2A-2C. In some implementations, image capture device(s) 1012 can be incorporated in a device comprising the processing or computing circuitry 1000 and can interface through the locate interface 1009.

Stored in the memory 1006 can be both data and several components that are executable by the processor 1003. In particular, stored in the memory 1006 and executable by the processor 1003 can be an HDR imaging program 1015 and potentially other application program(s). Also stored in the memory 1006 may be a data store 1018 and other data. In addition, an operating system 1021 may be stored in the memory 1006 and executable by the processor 1003. The memory is defined herein as including both volatile and nonvolatile memory and data storage components. Volatile components are those that do not retain data values upon loss of power. Nonvolatile components are those that retain data upon a loss of power. Thus, the memory 1006 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, optical disc such as compact disc (CD) or digital versatile disc (DVD), magnetic tapes accessed via an appropriate tape drive, holographic storage, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1003 may represent multiple processors 1003 and/or multiple processor cores (e.g., of a graphics processing unit), and the memory 1006 may represent multiple memories 1006 that operate in parallel processing circuits, respectively. In such a case, the local interface 1009 may be an appropriate network that facilitates communication between any two of the multiple processors 1003, between any processor 1003 and any of the memories 1006, or between any two of the memories 1006, etc. The local interface 1009 may comprise additional systems designed to coordinate this communication, including, for example, performing load balancing. The processor 1003 may be of electrical or of some other available construction.

A number of software components can be stored in the memory 1006 and can be executable by the processor 1003. An executable program may be stored in any portion or component of the memory 1006. In this respect, the term “executable” means a program file that is in a form that can ultimately be run by the processor 1003. Examples of executable programs may be, for example, a compiled program that can be translated into machine code in a format that can be loaded into a random access portion of the memory 1006 and run by the processor 1003, source code that may be expressed in proper format such as object code that is capable of being loaded into a random access portion of the memory 1006 and executed by the processor 1003, or source code that may be interpreted by another executable program to generate instructions in a random access portion of the memory 1006 to be executed by the processor 1003, etc. In particular, stored in the memory and executable by the processor can be a vehicle category classification program, an operating system and potentially other applications. Also stored in the memory may be a data store and other data. It is understood that there may be other applications that are stored in the memory and are executable by the processor as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

Although the HDR imaging program 1015 and other application program(s) or systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein. Also, any logic or application described herein, including the HDR imaging program 1015, that comprises software or code can be embodied in any non-transitory computer-readable medium for use by or in connection with an instruction execution system such as, for example, a processor 1003 in a computer system or other processing circuitry, device or system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The automated fine-grained vehicle classification using combined semantic and geometric features extracted from surveillance videos program, which comprises an ordered listing of executable instructions for implementing logical functions, can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can contain, store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM or Flash memory) (electronic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory. In addition, the scope of the certain embodiments of the present disclosure includes embodying the functionality of the preferred embodiments of the present disclosure in logic embodied in hardware or software-configured mediums.

### DISCUSSION

In summary, a portable widefield fundus camera with nonmydriatic HDR imaging capability is demonstrated herein. The HDR fundus camera employed miniaturized indirect ophthalmoscopy illumination to enable the portable design for widefield fundus photography. NIR light was used for image focusing and guidance. Pulsed visible light illumination with flexible power control allowed rapid, nonmydriatic imaging, within a time window before visible light illumination caused pupillary response.

To overcome the limitation of FOV in traditional fundus cameras with transpupillary illumination, trans-pars-planar and trans-palpebral illumination have been demonstrated to increase the FOV up to the ora serrata, i.e., the far end of the retina. Nevertheless, the illumination efficiency depends on the light wavelength, illumination location and the pigmentation level of the subject. The trans-pars-planar and trans-palpebral illumination and imaging protocols for clinical deployment can be standardized. Miniaturized indirect illumination has been demonstrated as an alternative illumination strategy for developing widefield portable fundus camera. However, the illumination reflectance artifact and light efficiency inhomogeneity limit the fundus image contrast. By rotating the ophthalmic lens, two frames can be captured with lens artifacts in different locations, and these two frames can be merged to get an artifact free image. The moving components used in the system hindered its application as a portable device. Herein orthogonal polarization illumination and imaging control are employed to eliminate the back reflected light. Since there was no moving component in the system, the portable fundus camera design can be readily achieved with simplified indirect illumination.

HDR imaging has been well-established in the field of digital imaging to expand the dynamic range and enhance the contrast of images. For example, most of the smartphone cameras have such HDR imaging option. However, the HDR imaging has not been previously reported in fundus cameras. For regular imaging situations, the exposure time periods can be flexibly controlled to optimize the visibility of low and high brightness components in sub-frames for subsequent HDR processing. However, for nonmydriatic fundus imaging, the available acquisition time is limited due to pupillary response caused by visible light illumination. NIR light guidance and rapid visible light power control were combined to meet the requirement of nonmydriatic fundus imaging. With the demonstrated HDR function, the portable widefield fundus camera showed superior capability to reveal pathological markers such as microaneurysms caused by DR (FIGS. 6A-6C), and pigment clumping and RPE atrophy caused by AMD (FIGS. 7A-7C), compared to a commercial portable fundus camera Volk Pictor Plus (Volk, Mentor, OH).

It should be noted that Volk Pictor Plus uses a broadband white light source. The light attenuation, due to scattering and absorption in biological tissues, is known to be wavelength dependent. For fundus imaging, the short wavelength, such as blue or green, light has a much lower efficiency, compared to that of red light with relatively long wavelength, Therefore, the images taken with Volk Pictor Plus are consistently red dominant (FIG. 6B and FIG. 7B). In contrast, the light source used in an exemplary system is a LED that has center wavelength at 565 nm (M565D2, Thorlabs Inc., Newton, NJ, USA) and 104 nm bandwidth (FWHM). In other words, the LED in the prototype system is green light dominated to optimize the visibility of retinal structure.

For comprehensive eye examination, fundus imaging of both central and peripheral regions is important. The demonstrated portable HDR fundus camera has a 101° eye-angle (67° visual angle) snapshot FOV for nonmydriatic fundus photography. In coordination with a fixation target, the FOV could be extended up to 190° eye-angle (134° visual-angle) horizontally and 146° eye-angle (100° visual-angle) vertically to visualize the retinal periphery for comprehensive eye examination. Comparative imaging and analysis were conducted with the portable HDR fundus camera and an ultra-widefield SLO Optomap imager to evaluate the FOV and image contrast. As shown in FIG. 8A, the portable HDR fundus camera showed similar imaging performance, and even better capability to reach peripheral fundus in the vertical direction. The widefield SLOs have been well established for improved clinical management of eye diseases, compared to traditional fundus cameras. However, the high cost and bulky design make them challenging for telemedicine application, which is particularly important for rural and underserved areas with limited access to experienced ophthalmologists and expensive medical devices. A low-cost, portable, wide field fundus camera may offer a unique opportunity to foster telemedicine ophthalmology to reduced is parities of medical care in rural and underserved areas.

Although the HDR function is demonstrated with a lab prototype nonmydriatic device, the same HDR function can be implemented with clinical fundus cameras. In principle, multiple images with different light exposures are needed to selectively capture the details in different fundus regions for following HDR processing. For nonmydriatic fundus photography, all images should be captured before the pupillary response caused by visible light illumination. For mydriatic fundus photography, the needed image-set can be readily acquired with either illumination exposure time or power control and after subsequent registering, HDR fundus image can be generated.

A portable, widefield fundus camera with nonmydriatic HDR imaging capability has been demonstrated and validated with both normal healthy and pathologic eyes. Miniaturized indirect ophthalmoscopy illumination was employed to achieve wide FOV, and orthogonal polarization control was used to eliminate illumination reflectance artifacts. Flash bracketed image acquisition and HDR processing were validated to implement high contrast fundus imaging. The portable HDR fundus camera provided superior capability to reveal pathological markers, compared to a conventional fundus camera. Because of the contrast improvement with HDR function, the fundus image contrast is comparable to the SLO Optomap imager. This portable, widefield, nonmydriatic HDR fundus camera promises a unique solution to facilitate affordable telemedicine. Other embodiments constructed in accordance with the principles of the present disclosure are contemplated as well.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A fundus camera, comprising:
a camera lens;
an imaging sensor positioned on a first side of the camera lens;
an ophthalmic lens positioned on a second side of the camera lens; and
a source of input light, the source configured to direct the input light through the ophthalmic lens to illuminate a retina of an eye of a subject, the source configured to illuminate the retina at sequentially increasing illumination power levels during a series of illumination periods within a pupillary reflex time of the eye, the series of illumination periods comprising at least three illumination periods prior to the pupillary reflex time of the eye;
where the imaging sensor is configured to capture a corresponding low dynamic range (LDR) image of output light reflected or backscattered by the retina during each of the series of illumination periods and received by the imaging sensor via the ophthalmic lens and camera lens.

2. The fundus camera of claim 1, wherein the sequentially increasing illumination power levels are doubled in power at each subsequent illumination period in the series of illumination periods within the pupillary reflex time.

3. The fundus camera of claim 2, wherein the series of illumination periods comprises at least two periods.

4. The fundus camera of claim 3, wherein the series of illumination periods comprises three periods with the sequentially increasing illumination power levels at 2 mW, 4 mW and 8 mW at the pupil plane for the subject.

5. The fundus camera of claim 1, wherein each illumination period is about 45 ms or less.

6. The fundus camera of claim 1, wherein the source is a source of linearly polarized input light with a polarization axis in a first direction, and
the fundus camera further comprises:
a quarter waveplate positioned on a side of the ophthalmic lens opposite the camera lens, the source configured to direct the linearly polarized input light to the quarter waveplate through the ophthalmic lens, the quarter waveplate converting the linearly polarized input light to circularly polarized input light that illuminates the retina at the increasing illumination levels during the series of illumination periods; and
a linear polarizer disposed between the ophthalmic lens and the camera lens, the linear polarizer configured to receive depolarized output light reflected or backscattered by the retina and linearly polarized output light converted by the quarter waveplate from helically flipped output light reflected by the retina, and allow polarized output light with a polarization axis in a second direction that is aligned with the linear polarizer to reach the imaging sensor via the camera lens.

7. The fundus camera of claim 6, wherein the source of the linearly polarized input light comprises a light source and a corresponding linear polarizer disposed between the light source and the ophthalmic lens, the light source and the camera lens positioned in a common plane with the light source offset from the camera lens by a buffer distance.

8. The fundus camera of claim 1, wherein the light source is configured to provide near infrared (NIR) light and visible light.

9. The fundus camera of claim 8, wherein the visible light is provided as the input light for illumination of the retina and the NIR light is provided for focus and guidance of the fundus camera.

10. A fundus camera system, comprising:
the fundus camera of claim 1; and
processing circuitry configured to generate a high dynamic range (HDR) image of the retina by combining the corresponding LDR images captured during the series of illumination periods prior to the pupillary reflex time of the eye.

11. The fundus camera system of claim 10, wherein intensity values of pixels of the corresponding LDR images are combined based at least in part upon a weighting function associated with each pixel.

12. The fundus camera system of claim 10, further comprising an external fixation target visible to another eye of the subject.

13. The fundus camera system of claim 12, wherein the external fixation target is repositionable with respect to the fundus camera.

14. A method for high dynamic range (HDR) imaging of a retina, comprising:
directing input light from a source positioned on a first side of an ophthalmic lens to illuminate a retina of an eye on a second side of the ophthalmic lens, the retina illuminated at sequentially increasing illumination power levels during a series of illumination periods within a pupillary reflex time of the eye, the series of illumination periods comprising at least three illumination periods prior to the pupillary reflex time of the eye of a subject;
capturing, by an imaging sensor, a corresponding low dynamic range (LDR) image of output light reflected or backscattered by the retina during each of the series of illumination periods and received by the imaging sensor via the ophthalmic lens and a camera lens positioned on the first side of an ophthalmic lens; and
generating, by processing circuitry, a high dynamic range (HDR) image of the retina by combining the corresponding LDR images captured during the series of illumination periods prior to the pupillary reflex time of the eye.

15. The method of claim 14, wherein the sequentially increasing illumination power levels are doubled in power at each subsequent illumination period in the series of illumination periods within the pupillary reflex time.

16. The method of claim 15, wherein the retina is illuminated for a fixed time period during each illumination period.

17. The method of 14, wherein intensity values of pixels of the corresponding LDR images are combined based at least in part upon a weighting function associated with each pixel.

18. The method of claim 14, comprising:
directing near infrared (NIR) light from the source to illuminate the retina; and
focusing and aligning the imaging sensor for capture of the corresponding LDR images prior to directing the input light from the source.

19. The method of claim 14, further comprising:
positioning an external fixation target at a location visible to another eye of the subject, the location at a first visual angle away from a macula centered image;
with the other eye focused on the fixation target, directing input light from the source to illuminate the retina at the sequentially increasing illumination power levels during a second series of illumination periods within the pupillary reflex time of the eye, the second series of illumination periods comprising at least three illumination periods prior to the pupillary reflex time of the eye;
capturing, by the imaging sensor, a corresponding LDR image of output light reflected or backscattered by the retina during each of the second series of illumination periods;
generating, by the processing circuitry, a second HDR image of the retina by combining the corresponding LDR images captured during the second series of illumination periods prior to the pupillary reflex time of the eye; and
generating a widefield HDR image by combining the HDR image of the retina with the second HDR image.

20. The method of claim 19, further comprising:
repositioning the external fixation target at another location visible to the other eye of the subject, the other location at a second visual angle away from the macula centered image opposite the first visual angle;
with the other eye focused on the fixation target, directing input light from the source to illuminate the retina at the sequentially increasing illumination power levels during a third series of illumination periods within the pupillary reflex time of the eye, the third series of illumination periods comprising at least three illumination periods prior to the pupillary reflex time of the eye;
capturing, by the imaging sensor, a corresponding LDR image of output light reflected or backscattered by the retina during each of the third series of illumination periods;
generating, by the processing circuitry, a third HDR image of the retina by combining the corresponding LDR images captured during the third series of illumination periods prior to the pupillary reflex time of the eye; and
generating an ultra-widefield HDR image by combining the HDR image of the retina with the second and third HDR images.
